SEQUENCE LISTING (18 sequences)

SEQ ID NO: 1 - protein, 196 aa, Rattus norvegicus
MDVTIQHPWFKRALGPFYPSRLFDQFFGEGLFEYDLLPFLSSTISPYYRQSLFRTVLDSGISELMTHMWFVMHQPHAGNPKNNPGKVRSDRDKFVIFLDVKHFSPEDLTVKVLEDFVEIHGKHNERQDDHGYISREFHRRYRLPSNVDQSALSCSLSADGMLTFSGPKVQSGLDAGHSERAIPVSREEKPSSAPSS

SEQ ID NO: 2 - protein, 175 aa, Rattus rattus
MDIAIHHPWIRRPFFPFHSPSRLFDQFFGEHLLESDLFSTATSLSPFYLRPPSFLRAPSWIDTGLSEMRMEKDRFSVNLDVKHFSPEELKVKVLGDVIEVHGKHEERQDEHGFISREFHRKYRIPADVDPLTITSSLSSDGVLTVNGPRKQASGPERTIPITREEKPAVTAAPKK

SEQ ID NO: 3 - protein, 215 aa, Homo sapiens
METQAEQQELETLPTTKMAQTNPTPGSLGPWKITIYDQENFQGKRMEFTSSCPNVSERSFDNVRSLKVESGAWIGYEHTSFCGQQFILERGEYPRWDAWSGSNAYHIERLMSFRPICSANHKESKMTIFEKENFIGRQWEISDDYPSLQAMGWFNNEVGSMKIQSGAWVCYQYPGYRGYQYILECDHHGGDYKHWREWGSHAQTSQIQSIRRIQQ

SEQ ID NO: 4 - protein, 197 aa, Homo sapiens
MSSAPAPGPAPASLTLWDEEDFQGRRCRLLSDCANVCERGGLPRVRSVKVENGVWVAFEYPDFQGQQFILEKGDYPRWSAWSGSSSHNSNQLLSFRPVLCANHNDSRVTLFEGDNFQGCKFDLVDDYPSLPSMGWASKDVGSLKVSSGAWVAYQYPGYRGYQYVLERDRHSGEFCTYGELGTQAHTGQLQSIRRVQH

SEQ ID NO: 5 - protein, 52 aa, Rattus norvegicus
METQTVQRELETLPTTKMAQTNPMPGSMGPWKITIYDQENFQGKRMEFTSSC

SEQ ID NO: 6 - protein, 177 aa, Rattus sp.
ENFQGKRMEFTSSCPNVSERSFDNVRSLKVECGAWIGYEHTSFCGQQFILERGEYPRWDAWSGSNAYHIERLMSFRPICSANHKESKITIFEKENFIGRQWEICDDYPSLQAMGWFNNEVGSMKIQCGAWVCYQYPGYRGYQYILECDHHGGDYKHWREWGTHAQTSQIQSIRRIQQ

SEQ ID NO: 7 - protein, 196 aa, Rattus norvegicus
MTLQCTKSAGHWRVVVWDEEGFQGRRHEFTAECPSVLDLGFETVRSLKVLSGAWVGFEHAGFQGQQYVLERGDYPGWDAWGGNTAYPAERLTSFRPVACANHRDSRLTIFEQENFLGRKGELSDDYPSLQAMGWDGTEVGSFHVQSGAWVCSQFPGYRGFQYVLESDHHSGDYKHFREWGSHAHTFQVQSVRRIQQ

SEQ ID NO: 8 - protein, 205 aa, Rattus norvegicus
MASDHQTQAGKPQPLNPKIIIFEQENFQGHSHELSGPCPNLKETGMEKAGSVLVQAGPWVGYEQANCKGEQFVFEKGEYPRWDSWTSSRRTDSLSSLRPIKVDSQEHKIILYENPNFTGKKMEIVDDDVPSFHAHGYQEKVSSVRVQSGTWVGYQYPGYRGLQYLLEKGDYKDNSDFGAPHPQVQSVRRIRDMQWHQRGAFHPSS

SEQ ID NO: 9 - protein, 178 aa, Homo sapiens
MSKTGTKITFYEDKNFQGRRYDCDCDCADFHTYLSRCNSIKVEGGTWAVYERPNFAGYMYILPQGEYPEYQRWMGLNDRLSSCRAVHLPSGGQYKIQIFEKGDFSGQMYETTEDCPSIMEQFHMREIHSCKVLEGVWIFYELPNYRGRQYLLDKKEYRKPIDWGAASPAVQSFRRIVE

SEQ ID NO: 10 - DNA, 1271 bp, Rattus norvegicus
gaattccgccgggagagtacaggagatatcttgactggggggctgcaaacgccaaagttggctcttttagaagagtcatggatttttactgaggcattttgagactctacttttctcctctagaaactaataaaatatgtagcttcaccaaccagaacatggacgtcaccatccagcacccttggttcaagcgcgccctggggcccttctaccccagccgactgttcgaccagttcttcggcgagggcctttttgaatacgacctgctgcccttcctgtcttccaccatcagcccctactaccgccagtctctcttccgcacagtgttggactccggcatctctgagctcatgacccatatgtggtttgtaatgcaccaaccacatgctggaaaccccaagaacaaccccggcaaggtccgatctgaccgggacaagtttgtcatcttcttggatgtgaagcacttctctcctgaggacctcaccgtgaaggtactggaagatttcgtggagatccatggcaaacacaacgagaggcaggatgaccatggctacatttcccgtgaatttcaccgtcgctaccgtctgccttccaatgtggaccagtccgccctctcctgctccttgtctgcggatggcatgctgaccttctctggccccaaggtccagtctggcttggatgctggccacagcgagagggccattcccgtgtcacgggaggagaagcccagctcggcaccctcgtcctgagcaggcctcgccttggttgtcccctgaggcccctgatccatccagcccaggggccacagcaaagagtctgccttcctgacttcttttctttctctttgtttcctttccactttctcagagggctgaggatttgagagagtggcttaaagagcttggggggtcttggcctgagatggctgcgggttcagggtgacccaggctcaacaccagccggtcagagggaatgatggcattgaactcttaagatttcctgtcctcctggaaagtggcatcgagctctgctccaaaggcagagtgaatggtggctaaccaaccccaagagccctctgccaagcccctggatggcagcctcccaccccctttgcccacacttaccgcaggcgtatatgctgggctccaacagtccgcttctctcatgccctcttcctgtgactttctctactatgtagtatcgctcctggggaccctggtcacccatgagaatggagcccctggcagacaataaagagcaggtgcgaattc

SEQ ID NO: 11 - DNA, 689 bp, Rattus rattus
cggttagccatcatggacatagccatccaccacccctggatccggcgtcccttctttcctttccactccccaagccgcctctttgaccagttcttcggagagcacctgttggagtctgacctcttttctacagccacttccctgagccccttctaccttcggccaccctccttcctgcgggcacctagctggattgacactgggctctcagagatgcgtatggagaaggacaggttctctgtgaacctggacgtgaagcacttctctccagaggaactcaaagtcaaggttctgggagacgtgattgaggtgcacggcaagcacgaagagcgccaggacgaacatggcttcatctccagggagttccacaggaagtaccggatcccagccgacgtggatcctctcaccattacttcttccctgtcatcggatggagtcctcactgtgaatggaccaaggaaacaggcctctggccctgagcgcaccattcccatcacccgtgaagagaagcctgctgtcactgcagcccctaagaagtagattccctttcctcgttgcattttttaagacaaggaagtttcccatcagcgaatgaacatctgtgactagtgccgaagcttactaatgctaagggctggcccagattattaagctaataaaaaatatcgttcagcaaaaaaaaaaaaaaa

SEQ ID NO: 12 - DNA, 798 bp, Homo sapiens
gtaccagatggagacccaggctgagcagcaggagctggaaacccttccaaccaccaagatggctcagaccaaccctacgccggggtccctggggccatggaagataaccatctatgatcaggagaactttcagggcaagaggatggagttcaccagctcctgtccaaatgtctctgagcgcagttttgataatgtccggtccctgaaggtggaaagtggcgcctggattggttatgagcataccagcttctgtgggcaacagtttatcctggagagaggagaataccctcgctgggatgcctggagtgggagtaatgcctaccacattgagcgtctcatgtccttccgccccatctgttcagctaatcataaggagtctaagatgaccatctttgagaaggaaaactttattggacgccagtgggagatctctgacgactacccctccttgcaagccatgggctggttcaacaacgaagtcggctccatgaagatacaaagtggggcctgggtttgctaccaatatcctggatatcgtgggtatcagtatatcttggaatgtgaccatcatggaggagactataaacattggagagagtggggctctcatgcccagacttcgcagatccaatcgattcgccgaatccaacagtagctgattaaaagctccaagtacgataattcctcaagcatgagaccttgctaagcactctagaaagttttatgttctgctcacagacattgctttcaaatgttagctggtgaaatccacaataaacgtcatttaaaaaaaaaaaa

SEQ ID NO: 13 - DNA, 700 bp, Homo sapiens
ctcgcgtgccgcgctcacccacccgcggcatgagcagcgcccccgcgccgggcccggcgcccgccagcctcacgctctgggacgaggaggacttccagggccgtcgctgtcggctgctaagcgactgtgcgaacgtctgcgagcgcggaggcctgcccagggtgcgctcggtcaaggtggaaaacggcgtttgggtggcctttgagtaccccgacttccagggacagcagttcattctggagaagggagactatcctcgctggagcgcctggagtggcagcagcagccacaacagcaaccagctgctgtccttccggccagtgctctgcgcgaaccacaatgacagccgtgtgacactgtttgagggggacaacttccaaggctgcaagtttgacctcgttgatgactacccatccctgccctccatgggctgggccagcaaggatgtgggttccctcaaagtcagctccggagcgtgggtggcctaccagtacccaggctaccgaggctaccagtatgtgttggagcgggaccggcacagcggagagttctgtacttacggtgagctcggcacacaggcccacactgggcagctgcagtccatccggagagtccagcactaggctccacggccccagacaccttccctgaggacactcaataaaggttcctgaatcttcctgccaaaaaaaaaaaaaaa

SEQ ID NO: 14 - DNA, 161 bp, Rattus norvegicus
accagatggagacccagactgtgcagcgggagctggaaactcttccaaccaccaagatggctcagaccaaccctatgccagggtccatggggccatggaagataaccatctatgatcaggagaacttccagggcaagaggatggagttcaccagctcctgc

SEQ ID NO: 15 - DNA, 650 bp, Rattus sp.
gagaacttccagggcaagaggatggagttcaccagctcctgcccaaatgtctctgaacgtagttttgataatgtccggtcacttaaggtggagtgtggcgcctggattggttatgagcacaccagcttctgtgggcaacagttcatcctggagagaggagaataccctcgatgggatgcctggagtgggagcaatgcctatcatattgagcgtctcatgtccttccgacccatctgttctgctaatcataaagagtctaagatcaccatctttgagaaagagaactttattggacgccagtgggagatctgtgatgactacccttccttacaagccatgggttggttcaacaatgaagtgggttccatgaagatacagtgtggagcttgggtttgctaccagtaccctggatatcgtggttatcagtatatcttggaatgtgaccatcacggaggagactacaaacactggagagagtggggaactcatgctcagacttctcagatccaatcaattcgccgaatacaacaatagtagagtcaaagattcaaaatcctcaagcatgcaacttactaagcattatagaattttatgttctgcccagacactgcatccaaatgttagcactaaaatccacaataaatgtcatt

SEQ ID NO: 16 - DNA, 800 bp, Rattus norvegicus
acatgtgccctgggcccttcttggactgagccaacatgaccctgcagtgcaccaagtcagctggacactggagggtggtggtgtgggacgaagaaggcttccagggtcgtcggcatgaattcacagctgagtgtcccagcgtgctggatctcggctttgagaccgtgcgatcactgaaagtcctgagcggagcgtgggtgggctttgagcacgctggcttccaagggcagcaatatgtgctggagaggggcgattacccaggctgggacgcctggggtggcaacacagcctaccccgccgagaggctcacctccttccggcctgtggcctgcgctaaccaccgcgactcaaggctgaccatcttcgagcaggagaacttcctgggcaggaaaggcgagctgagtgacgactatccctctctgcaggccatgggctgggacggcactgaagtgggctccttccatgttcagtctggcgcgtgggtttgttcccagtttcctggttaccgaggttttcagtacgtgctggagagcgatcaccactcgggtgactacaagcacttcagggagtggggctcccatgcccacaccttccaggtgcagagtgtgcgcaggatccagcagtgagagcgcacaagtgacctggggtgcctgcaaaacgactgagtgactggccggaggatgtggctgctcttggttctggctacccctgtgtcctctgggaaccccacacccctgtcagccagcccattccctgccagctcacgaagctcaaagaaataaaattaaaaacaaaagact

SEQ ID NO: 17 - DNA, 735 bp, Rattus norvegicus
gtcacctcgacaccagagagtccaccatggcctcagaccaccagacacaggcgggcaagccccagccccttaaccctaagatcatcatcttcgaacaggagaacttccagggccattcccatgagctcagcgggccctgccccaacctgaaggagactgggatggagaaggcaggctccgtcctggtgcaggctggaccctgggtgggctacgagcaggctaattgcaagggagagcagtttgtgtttgagaagggcgagtacccacgctgggactcctggaccagcagccggaggacggactccctcagttctctgagacccatcaaagtggacagccaggagcacaagatcatcttatatgagaaccccaactttacgggcaagaagatggagattgtcgatgatgacgtacccagctttcacgctcacggataccaagaaaaagtgtcttccgtgcgcgtgcagagcggcacgtgggttgggtatcagtaccctggctaccgtgggctgcagtatctgctggagaagggggattacaaggacaacagcgacttcggggcccctcacccccaggtgcaatctgtgcgtcgcatccgtgacatgcagtggcaccagcggggcgccttccacccctccagctaaagccctgcccctccctccttcccagggtccaggccagccaccggggtcctcctgacacccagagtgaataaagtgtggcttgcaacttgaa

SEQ ID NO: 18 - DNA, 698 bp, Homo sapiens
ctgaatttctttcagcactgggaaaaccagtctatgcaccaaaaatgtctaaaactggaaccaagattactttctatgaagacaaaaattttcaaggccgtcgctatgactgtgattgcgactgtgcagatttccacacatacctaagtcgctgcaactccattaaagtggaaggaggcacctgggctgtttatgaaaggcccaactttgctgggtacatgtacatcttaccacagggagagtaccctgaataccagcgttggatgggcctcaacgaccgcctcagctcctgcagagctgttcatctgcctagtggaggccagtataagattcagatctttgagaaaggggattttagtggtcagatgtatgaaaccaccgaagattgcccttccatcatggagcaatttcacatgcgagagatccactcctgtaaggtgctggagggtgtctggattttctatgagctacccaactaccgtggcaggcagtacctcctggacaagaaggagtaccggaagcccatcgattggggtgcagcctccccagctgtccagtctttccgccgcattgtggagtaatgacatgaatggggccatattcttcctggggcccaaatgctggctggccttgtggtccaaataggcatcatcaataaaacagttggcatgcatcccaaaaaaaaaaaaaaaaaaaaa

(19) United States
(12) Patent Application Publication
(10) Pub. No.: US 2009/0048146 A1
(43) Pub. Date: Feb. 19, 2009

(54) USE OF AGENTS THAT UPREGULATE CRYSTALLIN EXPRESSION IN THE RETINA AND OPTIC NERVE HEAD

(75) Inventors: Abot F. Clark, Arlington, TX (US); John C. Morrison, Portland, OR (US); Elaine C. Johnson, Portland, OR (US)

Correspondence Address: ALCON, IP LEGAL, TB4-8, 6201 SOUTH FREEWAY, FORT WORTH, TX, 76134, US

(73) Assignee: ALCON, INC., Hunenberg (CH)

(21) Appl. No.: 11/963,571

(22) Filed: Dec. 21, 2007

Related U.S. Application Data
(60) Provisional application No. 60/871,313, filed on Dec. 21, 2006.

(51) Int. Cl. A61K 38/02; C12Q 1/02; C12Q 1/68; G01N 33/00; C12N 15/11

(52) U.S. Cl. 514/2; 435/29; 435/6; 436/86; 435/320.1

(57) ABSTRACT

The present invention relates to methods to treat and/or prevent optic nerve damage in a subject by administering a composition comprising a crystallin agonist.

### RELATED APPLICATION

The present application claims priority to U.S. Provisional Patent Application No. 60/871,313 filed Dec. 21, 2006.

### TECHNICAL FIELD

The present invention relates to the use of agents that upregulate the expression of crystallin in the retina and optic nerve thereby treating and/or preventing glaucomatous optic neuropathy and retinal damage due to glaucoma.

### BACKGROUND OF THE INVENTION

Glaucomatous optic neuropathy (glaucoma) is a disease characterized by the permanent loss of visual function due to irreversible damage to the optic nerve. The several morphologically or functionally distinct types of glaucoma are typically characterized by elevated intraocular pressure (IOP), which is considered to be causally related to the pathological course of the disease. Examples include primary open angle glaucoma and angle closure glaucoma.

Ocular hypertension is a condition wherein IOP is elevated, but no apparent loss of visual function has occurred; such patients are considered to be at high risk for the eventual development of the vision loss associated with glaucoma. Some patients with glaucomatous field loss have relatively low IOP. These normal tension or low tension glaucoma patients can also benefit from agents that lower and control IOP. If glaucoma or ocular hypertension is detected early and treated promptly with medications that effectively reduce elevated intraocular pressure, the loss of visual function or its progressive deterioration can generally be reduced. Drug therapies that have proven to be effective for the reduction of intraocular pressure include both agents that decrease aqueous humor production and agents that increase the outflow facility. Continuously elevated IOP has been associated with the progressive deterioration of the retina and the loss of visual function. Pathological changes in the retina associated with glaucoma include changes in the neuroretinal rim size and shape, retinal nerve fiber layer loss, presence of parapapillary atrophy, and presence of retinal or optic disc hemorrhages.

Therefore, lowering IOP can be an objective for the treatment of glaucoma patients, in order to decrease the potential for, or severity of, vision loss. Unfortunately, many individuals do not respond well when treated with existing glaucoma therapies.

Crystallins are the major structural proteins in the lens, but are increasingly being recognized to be present in other tissues including retina (Magabo 2000). Several studies have shown that alpha- and beta-crystallins have chaperone activity. Furthermore, both types of crystallins are phosphorylated through cAMP-dependent and cAMP-independent pathways (Kantorow, 1998), suggesting a possible signalling function. Although beta-crystallins have not been previously reported in degenerating retina, Jones et al. (1998) reported an increase in expression of alpha-crystallin at P18 in rdl retinas, a time-point after most rods have died.

Interestingly, alpha-crystallins inhibit oxidative stress induced apoptosis in RPE cells in culture (Alge 2002). In a lens epithelial cell line, alpha-crystallin was shown to prevent apoptosis by inhibiting caspase-3 activation (Li 2001).

However, in none of these reports were compounds or agents used or suggested to stimulate or up-regulate crystallin expression and/or activity for the treatment of optic nerve damage in subjects.

### BRIEF SUMMARY OF THE INVENTION

The present inventors have discovered that expression of several crystallins is down-regulated in glaucomatous rat and human retinas, which makes the retina more susceptible to further damage. Thus, the present invention is directed to increasing the expression of crystallins (CRYAB, CRYM, CRYGS, CRYBB, CRYBA), which will allow the retina and optic nerve head to resist further stress.

One embodiment of the present invention comprises a method of manufacturing a crystallin agonist comprising: (a) providing a candidate substance, for example, a small molecule, a protein or a nucleic acid molecule suspected of increasing crystallin expression or activity in ocular tissue; (b) selecting the crystallin agonist by assessing the ability of the candidate substance to increase crystallin expression or activity in ocular tissue; and (c) manufacturing the selected crystallin agonist. Once the agonist is identified, a pharmaceutical composition comprising an agonist admixed with a pharmaceutical carrier can be produced using well known formulations.

The method can further comprise providing in a cell or a cell-free system a crystallin polypeptide and the crystallin polypeptide is contacted with the candidate substance. The crystallin polypeptide is selected from the group consisting of SEQ. ID. NO. 1, SEQ. ID. NO. 2, SEQ. ID. NO. 3, SEQ. ID. NO. 4, SEQ. ID. NO. 5, SEQ. ID. NO. 6, SEQ. ID. NO. 7, SEQ. ID. NO. 8, and SEQ. ID. NO. 9.

In further embodiments, the method comprises providing a nucleic acid molecule that encodes the crystallin polypeptide. The nucleic acid is selected from the group consisting of CRYAA, CRYAB, CRYBB, CRYBA, CRYGS, and CRYM.

Another embodiment of the present invention comprises a method of treating and/or preventing optic nerve damage comprising administering to a subject an effective amount of a crystallin agonist admixed with a pharmaceutical carrier, wherein said amount increases expression and/or activity of the crystallin in ocular tissue thereby treating and/or preventing optic nerve damage. More particularly, optic nerve damage is glaucoma, for example, primary open angle glaucoma, or optic neuropathy. In certain embodiments, the subject that is being treated has increased intraocular pressure in at least one eye.

Another embodiment comprises an expression vector comprising: a) a nucleic acid sequence selected from the group consisting of SEQ. ID. NO. 10, SEQ. ID. NO. 11, SEQ. ID. NO. 12, SEQ. ID. NO. 13, SEQ. ID. NO. 14, SEQ. ID. NO. 15, SEQ. ID. NO. 16, SEQ. ID. NO. 17 and SEQ. ID. NO. 18; or b) an isolated polynucleotide sequence encoding a protein, wherein said protein is selected from the group consisting of: (1) a polynucleotide sequence encoding a sequence selected from the group consisting of SEQ. ID. NO. 1, SEQ. ID. NO. 2, SEQ. ID. NO. 3, SEQ. ID. NO. 4, SEQ. ID. NO. 5, SEQ. ID. NO. 6, SEQ. ID. NO. 7, SEQ. ID. NO. 8, and SEQ. ID. NO. 9; (2) a polynucleotide sequence encoding an amino acid sequence having at least 80% identity with a sequence selected from the group consisting of SEQ. ID. NO. 1, SEQ. ID. NO. 2, SEQ. ID. NO. 3, SEQ. ID. NO. 4, SEQ. ID. NO. 5, SEQ. ID. NO. 6, SEQ. ID. NO. 7, SEQ. ID. NO. 8, and SEQ. ID. NO. 9; (3) an isolated nucleic acid molecule that hybridizes with the polynucleotide sequence of (1) under hybridization conditions of 0.02 M to about 0.15 M NaCl at temperatures of about 50° C. to about 70° C.; and (4) an isolated polynucleotide sequence that is complementary to (1), (2) or (3).

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention when considered in connection with any accompanying figures. Figures provided herein are intended to help illustrate the invention or assist with developing an understanding of the invention, and are not intended to be definitions of the invention's scope.

### DESCRIPTION OF ILLUSTRATIVE EMBODIMENTS

### I. Definitions

Unless defined otherwise, technical and scientific terms used herein have the same meaning as commonly understood by one of ordinary skill in the art to which this invention belongs. For purposes of the present invention, the following terms are defined below.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having”, “including”, “containing” and “comprising” are interchangeable and one of skill in the art is cognizant that these terms are open ended terms.

The term “effective amount” as used herein is defined as an amount of the agent that will increase or enhance crystallin expression in retinal and/or optic nerve head tissue in a subject having glaucoma or a subject at risk for developing glaucoma. Thus, an effective amount is an amount sufficient to detectably and repeatedly ameliorate, reduce, minimize or limit the extent of the disease or its symptoms.

The term “intraocular pressure” or “IOP” as used herein refers to the pressure of the fluid inside the eye. In a normal human eye, IOP is typically in the range of 10 to 21 mm Hg. IOP varies among individuals, for example, it may become elevated due to anatomical problems, inflammation of the eye, as a side-effect from medication or due to genetic factors. “Elevated” intraocular pressure is usually considered to be ≧21 mm Hg, which is also considered to be a risk factor for the development of glaucoma. However, some individuals with an elevated IOP may not develop glaucoma and are considered to have ocular hypertension.

As used herein, the terms “identity” or “similarity”, as known in the art, are relationships between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Both identity and similarity can be readily calculated by known methods such as those described in: Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991. Methods commonly employed to determine identity or similarity between sequences include, but are not limited to those disclosed in Carillo, H., and Lipman, D., SIAM J Applied Math., 48:1073 (1988). Methods to determine identity and similarity are codified in publicly available computer programs. Preferred computer program methods to determine identity and similarity between two sequences include, but are not limited to, GCG program package, Devereux, J., et al., Nucleic Acids Research, 12(1), 387 (1984)), BLASTP, BLASTN, and FASTA Atschul, S. F. et al., J. Molec. Biol., 215, 403 (1990)).

The terms “glaucomatous optic neuropathy” or “glaucoma” are interchangeable. Glaucoma refers to a disease characterized by the permanent loss of visual function due to irreversible damage to the optic nerve. The two main types of glaucoma are primary open angle glaucoma (POAG) and angle closure glaucoma.

The term “optic nerve” as used herein refers to the nerve or cranial nerve II, which transmits visual information from the retina to the brain.

The term “optic nerve damage” as used herein refers to an alteration of the normal structure or function of the optic nerve. The alteration of the normal structure or function of the optic nerve may be the result of damage of the optic nerve, which may involve any disease, disorder or insult. Such diseases or disorders include, but are not limited, glaucoma. An alteration of the normal function of the optic nerve includes any that results in an alteration of the ability of the optic nerve to function appropriately, such as transmit visual information from the retina to the brain. An alteration in function may manifest itself, for example, as loss of visual field, impaired central visual acuity, abnormal color vision, and so forth. Examples of alteration of structure include nerve fiber loss in the retina, abnormal cupping of the optic nerve and pallor of the optic, swelling of the optic nerve. “Optic nerve damage” as used herein may include optic nerve damage to one or both optic nerves of a subject

As used herein, the term “susceptibility” or “suspected of having” refers to an individual or subject that is likely to develop glaucoma. For example, the subject may have elevated intraocular pressure in one or both eyes without any other findings associated with glaucoma. While such an individual does not clinically carry a diagnosis of glaucoma, such an individual is at risk of developing glaucoma by virtue of the presence of the elevation in intraocular pressure. For example, the intraocular pressure may be greater or equal to 21 mm Hg in one or both eyes. A subject without elevated intraocular pressure who does not have glaucoma may also be susceptible to the development of glaucoma. For example, the subject may have a family history of glaucoma. The subject may or may not have a family history of glaucoma. “Susceptibility” is determined and assessed by any method known to those of ordinary skill in the art. For example, susceptibility can be determined based on results of physical examination, family history, or genetic screening techniques well-known to those of ordinary skill in the art.

As used herein, the term “stimulator” or “agonist” is defined as a compound or composition that enhances the activity of crystallin. The enhanced activity can be crystallin gene activity or crystallin protein activity. A stimulator can be a polynucleotide, a polypeptide, an antibody, or a small molecule.

The terms “treatment” and “treating” refer to administration or application of a therapeutic agent to a subject or performance of a procedure or modality on a subject for the purpose of obtaining a therapeutic benefit of a disease or health-related condition. Thus, one of skill in the art realizes that a treatment may improve the disease condition, but may not be a complete cure for the disease.

The term “therapeutic benefit” or “therapeutically effective” as used throughout this application refers to anything that promotes or enhances the well-being of the subject with respect to the medical treatment of his condition. This includes, but is not limited to, a reduction in the frequency or severity of the signs or symptoms of a disease. Therapeutic benefit also includes a reduction in intraocular pressure compared to intraocular pressure in the absence of the therapeutic agent. Therapeutic benefit also includes reducing the signs or symptoms associated with glaucoma in a subject with glaucoma. For example, a therapeutic benefit in a patient with glaucoma is obtained where there is no further progression of visual field loss in the affected eye, or a slowing of the rate of progression of visual field loss in the affected eye.

The terms “prevention” and “preventing” as used herein are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset of a disease or health-related condition. An individual with an eye that is at risk of developing glaucoma can be treated with a crystallin agonist as set forth herein for the purpose of blocking the onset of the signs or symptoms of glaucoma (i.e., prevention of glaucoma).

### II. Crystallins

Crystallins are known as lens proteins, however, they are also expressed in other tissues, for example the retina. Most crystallins belong to the family of small heat shock proteins (hsp20) and many function as molecular chaperones, thereby helping cells resist stress.

In certain embodiments, an agonist or stimulator of crystallin genes or proteins are administered to a subject to increase or augment the activity and/or expression of crystallin in ocular tissue, more particularly retinal and/or optic nerve tissue. The agonist of the present invention include, but are not limited to polynucleotides (RNA or DNA), polypeptides, peptides, peptide-like molecules, small molecules or other compositions that are capable of increasing or stimulating the activity and/or expression of crystallin. Yet further, the crystallin agonist may also include compounds that prevent the degradation of crystallin mRNA or gene products and/or enhance or increase the stability of crystallin gene products.

In further aspects, crystallin agonist may be screened using standard techniques in the art, which are more fully described elsewhere in this application. Examples of molecules that may be screened include, but are not limited to, small organic molecules, peptides or peptide-like molecules, nucleic acids, polypeptides, antibodies, peptidomimetics, carbohydrates, lipids or other organic (carbon-containing) or inorganic molecules. Many pharmaceutical companies have extensive libraries of chemical and/or biological mixtures, often fungal, bacterial, or algal extracts, which can be screened with any of the assays of the invention to identify compounds that increase or stimulate the activation and/or expression of crystallin. Further, in drug discovery, for example, proteins have been fused with antibody Fc portions for the purpose of high-throughput screening assays to identify potential modulators of new polypeptide targets (D. Bennett et al., 1995 and K. Johanson et al., 1995).

A. Nucleic Acids and Proteins

As used herein, the term “crystallin gene product” refers to a protein or polypeptide having an amino acid sequence that is substantially identical to a native crystallin amino acid sequence (or RNA, if applicable) or that is biologically active, in that it is capable of performing a functional activity similar to an endogenous crystallin and/or cross-reacting with anti-crystallin antibody raised against crystallin.

The terms “crystallin gene product” also include related-compounds of the respective molecules that exhibit at least some biological activity in common with their native counterparts. Such related-compounds include, but are not limited to, fragments or truncated polypeptides and polypeptides having fewer amino acids than the native polypeptide or polypeptides having at least 70%, at least 75%, at least 80%, at least 85%, at least 90%, at least 95% or at least 99% percent identity or similarity to native crystallin polypeptide sequences. The native crystallin polypeptide sequences include, but are not limited to CRYAA (SEQ. ID. NO. 1, GenBank accession AAA93366), CRYAB (SEQ. ID. NO. 2, GenBank accession No. CAA42910), CRYBA1 (SEQ. ID. NO. 3; GenBank accession No. NP_{—}005199), CRYBA2 (SEQ. ID. NO. 4, GenBank accession No. NP_{—}005200), CRYBA3 (SEQ. ID. NO. 5, GenBank accession No. AAB67118), CRYBA1/3 (SEQ. ID. NO. 6, GenBank accession No. CAA33241), CRYBA4 (SEQ. ID. NO. 7, GenBank accession No. AAB67117), CRYBB2 (SEQ. ID. NO. 8, GenBank accession No. CAA34204), CRYGS (SEQ. ID. NO. 9, GenBank accession No. AAD45901).

The term “crystallin gene” “crystallin polynucleotide” or “crystallin nucleic acid” refers to at least one molecule or strand of DNA (e.g., genomic DNA, cDNA) or RNA sequence (antisense RNA, siRNA, shRNA) a derivative or mimic thereof, comprising at least one nucleotide base, such as, for example, a naturally occurring purine or pyrimidine base found in DNA (e.g., adenine “A,” guanine “G,” thymine “T,” and cytosine “C”) or RNA (e.g., A, G, uracil “U,” and C). The term “nucleic acid” encompasses the terms “oligonucleotide” and “polynucleotide.” These definitions generally refer to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. An “isolated nucleic acid” as contemplated in the present invention may comprise transcribed nucleic acid(s), regulatory sequences, coding sequences, or the like, isolated substantially away from other such sequences, such as other naturally occurring nucleic acid molecules, regulatory sequences, polypeptide or peptide encoding sequences, etc.

More particularly, a “crystallin gene or crystallin polynucleotide” may also comprise any combination of associated control sequences, as well related-sequences, such as fragments, of the respective molecules that exhibit at least some biological activity in common with their native counterparts. Biological activity similar to the native counterparts will include coding for a crystallin protein that has activity that reduces stress of the retina and the optic nerve thereby treating or preventing glaucoma. The native crystallin polynucleotide sequences include, but are not limited to CRYAA (SEQ. ID. NO. 10, GenBank accession U47921), CRYAB (SEQ. ID. NO. 11, GenBank accession No. X60351), CRYBA1 (SEQ. ID. NO. 12; GenBank accession No. NM_{—}005208), CRYBA2 (SEQ. ID. NO. 13, GenBank accession No. NM_{—}005209), CRYBA3 (SEQ. ID. NO. 14, GenBank accession No. AF013248), CRYBA1/3 (SEQ. ID. NO. 15, GenBank accession No. X15143), CRYBA4 (SEQ. ID. NO. 16, GenBank accession No. AF013247), CRYBB2 (SEQ. ID. NO. 17, GenBank accession No. X16072), and CRYGS (SEQ. ID. NO. 18, GenBank accession No. AF101703). Thus, nucleic acid compositions encoding crystallin are herein provided and are also available to a skilled artisan at accessible databases, including the National Center for Biotechnology Information's GenBank database and/or commercially available databases, such as from Celera Genomics, Inc. (Rockville, Md.). Also included are splice variants that encode different forms of the protein, if applicable. The nucleic acid sequences may be naturally occurring or synthetic.

Still further, the “crystallin nucleic acid sequence,” “crystallin polynucleotide,” and “crystallin gene product” refer to nucleic acids provided herein, analogs thereof, homologs thereof, and sequences having substantial similarity and function, respectively. The term “substantially identical”, when used to define either a crystallin amino acid sequence or crystallin polynucleotide sequence, means that a particular subject sequence, for example, a mutant sequence, varies from the sequence of natural crystallin, respectively, by one or more substitutions, deletions, or additions, the net effect of which is to retain at least some of the biological activity found in the native crystallin protein, respectively. Alternatively, DNA analog sequences are “substantially identical” to specific DNA sequences disclosed herein if: (a) the DNA analog sequence is derived from coding regions of the natural crystallin gene, respectively; or (b) the DNA analog sequence is capable of hybridization to DNA sequences of crystallin under moderately stringent conditions and crystallin, respectively having biological activity similar to the native proteins; or (c) DNA sequences which are degenerative as a result of the genetic code to the DNA analog sequences defined in (a) or (b). Substantially identical analog proteins will be greater than about 80% similar to the corresponding sequence of the full-length native protein, more preferably, greater than 90% similar to the corresponding sequence of the full-length native protein, and most preferably, greater than 95% to the corresponding sequence of the full-length native protein. Sequences having lesser degrees of similarity but comparable biological activity are considered to be equivalents. Comparable biological activity would include the ability to produce a functional crystallin polypeptide that is capable of reducing stress in the retina and optic nerve head thereby treating and/or preventing glaucoma. In determining polynucleotide sequences, all subject polynucleotide sequences capable of encoding substantially similar amino acid sequences are considered to be substantially similar to a reference polynucleotide sequence, regardless of differences in codon sequence.

As used herein, “hybridization”, “hybridizes” or “capable of hybridizing” is understood to mean the forming of a double or triple stranded molecule or a molecule with partial double or triple stranded nature. The term “hybridization”, “hybridize(s)” or “capable of hybridizing” encompasses the terms “stringent condition(s)” or “high stringency” and the terms “low stringency” or “low stringency condition(s)” or “moderately stringent conditions”.

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. For example, a medium or moderate stringency condition could be provided by about 0.1 to 0.25 M NaCl at temperatures of about 37° C. to about 55° C. Under these conditions, hybridization may occur even though the sequences of probe and target strand are not perfectly complementary, but are mismatched at one or more positions. In another example, a low stringency condition could be provided by about 0.15 M to about 0.9 M salt, at temperatures ranging from about 20° C. to about 55° C. Of course, it is within the skill of one in the art to further modify the low or high stringency conditions to suit a particular application. For example, in other embodiments, hybridization may be achieved under conditions of, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

Naturally, the present invention also encompasses nucleic acid sequences that are complementary, or essentially complementary, to the sequences set forth herein, for example, in SEQ. ID. NO. 10, SEQ. ID. NO. 11, SEQ. ID. NO. 12, SEQ. ID. NO. 13, SEQ. ID. NO. 14, SEQ. ID. NO. 15, SEQ. ID. NO. 16, SEQ. ID. NO. 17 and/or SEQ. ID. NO. 18. Nucleic acid sequences that are “complementary” are those that are capable of base-pairing according to the standard Watson-Crick complementarity rules. As used herein, the terms “complementary sequences” and “essentially complementary sequences” means nucleic acid sequences that are substantially complementary to, as may be assessed by the same nucleotide comparison set forth above, or are able to hybridize to a nucleic acid segment of one or more sequences set forth herein. Such sequences may encode an entire crystallin molecule or functional or non-functional fragments thereof.

In certain embodiments, a “complementary” nucleic acid comprises a sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

B. Transcription Factors

Transcription factors are regulatory proteins that bind to a specific DNA sequence (i.e., promoters and enhancers) and regulate transcription of an encoding DNA region. Typically, a transcription factor comprises a binding domain that binds to DNA (a DNA binding domain) and a regulatory domain that controls transcription. Where a regulatory domain activates transcription, that regulatory domain is designated an activation domain. Where that regulatory domain inhibits transcription, that regulatory domain is designated a repression domain.

Activation domains, and more recently repression domains, have been demonstrated to function as independent, modular components of transcription factors. Activation domains are not typified by a single consensus sequence but instead fall into several discrete classes: for example, acidic domains in GAL4 (Ma, et al. 1987), GCN4 (Hope, et al., 1987), VP16 (Sadowski, et al. 1988), and GATA-1 (Martin, et al. 1990); glutamine-rich stretches in Sp1 (Courey, et al. 1988) and Oct-2/OTF2 (Muller-Immergluck, et al. 1990; Gerster, et al. 1990); proline-rich sequences in CTF/NF-1 (Mermod, et al. 1989); and serine/threonine-rich regions in Pit-1/GH-F-1 (Theill, et al. 1989) all function to activate transcription. The activation domains of fos and jun are rich in both acidic and proline residues (Abate, et al. 1991; Bohmann, et al. 1989); for other activators, like the CCAAT/enhancer-binding protein C/EBP (Friedman, et al. 1990), no evident sequence motif has emerged.

In the present invention, it is contemplated that transcription factors can be used to stimulate or increase the expression of a crystallin gene.

C. Expression Vectors

The present invention may involve using expression constructs as the pharmaceutical compositions. In certain embodiments, it is contemplated that the expression construct comprises one or more polynucleotide sequences encoding polypeptides which can act as agonists of crystallin and/or crystallin-related compounds. One of skill in the art would be able to determine, depending upon the desired usage of the expression construct, whether the polynucleotide sequences should encode a polypeptide that functions as an agonist of crystallin (therapeutic protocols).

In certain embodiments, the present invention involves the manipulation of genetic material to produce expression constructs that encode agonists of crystallin and/or crystallin-related compounds. Thus, the crystallin agonist and/or related-compound is contained in an expression vector. Such methods involve the generation of expression constructs containing, for example, a heterologous nucleic acid sequence encoding an agonist of interest and a means for its expression, replicating the vector in an appropriate cell, obtaining viral particles produced therefrom, and infecting cells with the recombinant virus particles.

As used in the present invention, the term “expression vector” refers to any type of genetic construct comprising a nucleic acid coding for crystallin agonist and/or related compounds. In some cases, DNA molecules are then translated into a protein, polypeptide, or peptide. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra. It is contemplated in the present invention, that virtually any type of vector may be employed in any known or later discovered method to deliver nucleic acids encoding an agonist of crystallin or related molecules. Where incorporation into an expression vector is desired, the nucleic acid encoding a crystallin agonist or related molecule may also comprise a natural intron or an intron derived from another gene. Such vectors may be viral or non-viral vectors as described herein, and as known to those skilled in the art. An expression vector comprising a nucleic acid encoding a crystallin agonist or related molecule may comprise a virus or engineered construct derived from a viral genome.

In particular embodiments of the invention, a plasmid vector is contemplated for use to transfect a host cell. In general, plasmid vectors containing replicon and control sequences that are derived from species compatible with the host cell are used in connection with these hosts. The vector ordinarily carries a replication site, as well as marking sequences that are capable of providing phenotypic selection in transformed cells. Plasmid vectors are well known and are commercially available. Such vectors include, but are not limited to, pWLNEO, pSV2CAT, pOG44, PXT1, pSG (Stratagene) pSVK3, pBSK, pBR322, pUC vectors, vectors that contain markers that can be selected in mammalian cells, such as pcDNA3.1, episomally replicating vectors, such as the pREP series of vectors, pBPV, pMSG, pSVL (Pharmacia), adenovirus vector (AAV; pCWRSV, Chatterjee et al. (1992)); retroviral vectors, such as the pBABE vector series, a retroviral vector derived from MoMuLV (pG1Na, Zhou et al., (1994)); and pTZ18U (BioRad, Hercules, Calif.).

In one embodiment, a gene encoding a crystallin agonist or structural/functional domain thereof or a crystallin-related compound is introduced in vivo in a viral vector. The ability of certain viruses to enter cells via receptor-mediated endocytosis and to exist as episomal elements or integrate into the host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). Such vectors include an attenuated or defective DNA virus, such as but not limited to herpes simplex virus (HSV), papilloma virus, Epstein Barr virus (EBV), adenovirus, adeno-associated virus (AAV), lentivirus and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, any tissue can be specifically targeted. Examples of particular vectors include, but are not limited to, a defective herpes virus 1 (HSV1) vector (Kaplitt et al., 1991) an attenuated adenovirus vector, (Stratford-Perricaudet et al., 1992), and a defective adeno-associated virus vector (Samulski et al., 1987 and Samulski et al., 1989).

In another embodiment the gene can be introduced in a retroviral vector, e.g., as described in U.S. Pat. No. 5,399,346; Mann et al., 1983; U.S. Pat. No. 4,650,764; U.S. Pat. No. 4,980,289; Markowitz et al., 1988; U.S. Pat. No. 5,124,263; International Patent Publication No. WO 95/07358; and Kuo et al., 1993, each of which is incorporated herein by reference in its entirety. Targeted gene delivery is described in International Patent Publication WO 95/28494.

Alternatively, the vector can be introduced in vivo by lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker. The use of cationic lipids may promote encapsulation of negatively charged nucleic acids, and also promote fusion with negatively charged cell membranes. The use of lipofection to introduce exogenous genes into the specific organs in vivo has certain practical advantages. Molecular targeting of liposomes to specific cells represents one area of benefit. Lipids may be chemically coupled to other molecules for the purpose of targeting. Targeted peptides, e.g., hormones or neurotransmitters, and proteins such as antibodies, or non-peptide molecules could be coupled to liposomes chemically.

It is also possible to introduce the vector in vivo as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (Wu and Wu, 1988).

As used herein, the terms “cell,” “cell line,” and “cell culture” may be used interchangeably. Cell lines used in the present invention can include, but are not limited to retinal cell lines, such as retinal ganglion cells, R28 (immortalized retinal precursor cell line), and optic nerve cells. One of skill in the art is cognizant that in addition to commercially available cell lines, primary cultures of cells may also be used in the present invention. All of these terms also include their progeny, which is any and all subsequent generations formed by cell division. It is understood that all progeny may not be identical due to deliberate or inadvertent mutations. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, crystallin molecule. Therefore, recombinant cells are distinguishable from naturally occurring cells that do not contain a recombinantly introduced nucleic acid.

In certain embodiments, it is contemplated that nucleic acid or proteinaceous sequences may be co-expressed with other selected nucleic acid or proteinaceous sequences in the same host cell. Co-expression may be achieved by co-transfecting the host cell with two or more distinct recombinant vectors. Alternatively, a single recombinant vector may be constructed to include multiple distinct coding regions for nucleic acids, which could then be expressed in host cells transfected with the single vector.

A gene therapy vector as described above can employ a transcription control sequence operably associated with the sequence for the crystallin agonist or related compound inserted in the vector. Such an expression vector is particularly useful to regulate expression of a therapeutic crystallin agonist.

### III. Methods of Manufacturing Agonists

The present invention contemplates methods for manufacturing agonist or stimulators that affect the activity and/or expression of one or more crystallins. These methods may comprise random screening of large libraries of candidate substances; alternatively, the methods may be used to focus on particular classes of compounds selected with an eye towards structural attributes that are believed to make them more likely to modulate the function or activity or expression of crystallin.

By function, it is meant that one may assay for mRNA expression, protein expression, protein activity, or binding activity, and otherwise determine functions contingent on the crystallin proteins or nucleic acid molecules.

A. Agonists

The present invention further comprises methods for identifying, making, generating, providing, manufacturing or obtaining agonists of crystallin activity or expression. Crystallin nucleic acid or polypeptide may be used as a target in identifying compounds that increase or enhance crystallin activity or expression in ocular tissue, decrease or down-regulate retinal damage associated with elevated intraocular pressure or glaucoma, and/or decrease or slow the progression of glaucoma and other optic neuropathies. These assays may comprise random screening of large libraries of candidate substances; alternatively, the assays may be used to focus on particular classes of compounds selected with an eye towards structural attributes that are believed to make them more likely to inhibit the function of crystallin molecules. By function, it is meant that one may assay for stimulation or upregulation or enhancement of activity of crystallin in ocular tissue, or stimulation or enhancement of expression of crystallin, for example. Such assays may include, for example, a luciferase reportor system in which luciferase activity is measured.

To identify, make, generate, provide, manufacture or obtain a crystallin agonist, one generally will determine the activity of the crystallin molecule in the presence, absence, or both of the candidate substance, wherein an agonist is defined as any substance that up-regulates, increases, enhances, or stimulates crystallin expression or activity. For example, a method may generally comprise:

(a) providing a candidate substance suspected of increasing crystallin expression or activity;

(b) assessing the ability of the candidate substance to increase crystallin expression or activity;

(c) selecting a crystallin agonist; and

(d) manufacturing the agonist.

In further embodiments, a crystallin polypeptide or nucleic acid may be provided in a cell or a cell free system and the crystallin polypeptide or nucleic acid may be contacted with the candidate substance. Next, an agonist is selected by assessing the effect of the candidate substance on crystallin activity or crystallin expression. Upon identification of the agonist, the method may further provide the step of manufacturing of the agonist using well known techniques in the art, such as synthesizing the compound or deriving the compound from a natural source.

As used herein, the term “candidate substance” refers to any molecule that may potentially increase, stimulate or enhance crystallin activity, expression or function. Candidate compounds may include fragments or parts of naturally-occurring compounds or may be found as active combinations of known compounds which are otherwise inactive. The candidate substance can be a nucleic acid, a polypeptide, a small molecule, etc. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds.

One basic approach to search for a candidate substance is screening of compound libraries. One may simply acquire, from various commercial sources, small molecule libraries that are believed to meet the basic criteria for useful drugs in an effort to “brute force” the identification of useful compounds. Screening of such libraries, including combinatorially generated libraries, is a rapid and efficient way to screen a large number of related (and unrelated) compounds for activity. Combinatorial approaches also lend themselves to rapid evolution of potential drugs by the creation of second, third and fourth generation compounds modeled of active, but otherwise undesirable compounds. It will be understood that an undesirable compound includes compounds that are typically toxic, but have been modified to reduce the toxicity or compounds that typically have little effect with minimal toxicity and are used in combination with another compound to produce the desired effect.

In specific embodiments, a small molecule library that is created by chemical genetics may be screened to identify a candidate substance that may be a modulator of the present invention (Clemons et al., 2001; Blackwell et al., 2001). Chemical genetics is the technology that uses small molecules to modulate the functions of proteins rapidly and conditionally. The basic approach requires identification of compounds that regulate pathways and bind to proteins with high specificity. Small molecules are prepared using diversity-oriented synthesis, and the split-pool strategy to allow spatial segregation on individual polymer beads. Each bead contains compounds to generate a stock solution that can be used for many biological assays.

The most useful pharmacological compounds may be compounds that are structurally related to compounds which interact naturally with compounds that modulate crystallin transcription or activity. Creating and examining the action of such molecules is known as “rational drug design,” and include making predictions relating to the structure of target molecules. Thus, it is understood that the candidate substance identified by the present invention may be a small molecule activator or any other compound (e.g., polypeptide or polynucleotide) that may be designed through rational drug design starting from known agonists of crystallin.

The goal of rational drug design is to produce or manufacture structural analogs of biologically active target compounds. By creating such analogs, it is possible to fashion drugs which are more active or stable than the natural molecules, which have different susceptibility to alteration or which may affect the function of various other molecules. In one approach, one would generate a three-dimensional structure for a molecule similar to crystallin, and then design a molecule for its ability to interact with a crystallin-related molecule. This could be accomplished by X-ray crystallography, computer modeling or by a combination of both approaches. The same approach may be applied to identifying interacting molecules of crystallin.

It also is possible to use antibodies to ascertain the structure of a target compound or activator. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

It will, of course, be understood that all the screening methods of the present invention are useful in themselves notwithstanding the fact that effective candidates may not be found. The invention provides methods for screening for such candidates, not solely methods of finding them.

B. In Vitro Assays

A quick, inexpensive and easy assay to run is a binding assay. Binding of a molecule to a target (e.g., crystallin) may, in and of itself, be agonist, due to steric, allosteric or charge-charge interactions. This can be performed in solution or on a solid phase and can be utilized as a first round screen to rapidly eliminate certain compounds before moving into more sophisticated screening assays. In one embodiment of this kind, the screening of compounds that bind to crystallin molecules or fragments thereof are provided.

A target crystallin protein may be either free in solution, fixed to a support, expressed in or on the surface of a cell. Either the crystallin protein or the compound may be labeled, thereby indicating if binding has occurred. In another embodiment, the assay may measure the activation of crystallin to a natural or artificial substrate or binding partner. Competitive binding assays can be performed in which one of the agents is labeled. Usually, the target crystallin protein will be the labeled species, decreasing the chance that the labeling will interfere with the binding moiety's function. One may measure the amount of free label versus bound label to determine binding or activation of binding. These approaches may be utilized on crystallin molecules.

A technique for high throughput screening of compounds is described in WO 84/03564. Large numbers of small peptide test compounds are synthesized on a solid substrate, such as plastic pins or some other surface. The peptide test compounds are reacted with, for example, crystallin protein and washed. Bound polypeptide is detected by various methods.

C. In Cyto Assays

Various cell lines that express crystallin related proteins can be utilized for screening of candidate substances. For example, cells containing crystallin proteins with an engineered indicator can be used to study various functional attributes of candidate compounds. In such assays, the compound would be formulated appropriately, given its biochemical nature, and contacted with a target cell. This same approach may be utilized to study various functional attributes of candidate compounds that effect crystallin.

Depending on the assay, culture may be required. As discussed above, the cell may then be examined by virtue of a number of different physiologic assays (e.g., growth, size, or survival). Alternatively, molecular analysis may be performed in which the function of crystallin and crystallin related pathways may be explored. This involves assays such as those for protein production, enzyme function, substrate utilization, mRNA expression (including differential display of whole cell or polyA RNA) and others.

D. In Vivo Assays

The present invention particularly contemplates the use of various animal models. A wide variety of mouse, rat, rabbit, sat, dog, and monkey models of glaucoma have been reported, In addition, transgenic animals can be made by any known procedure, including microinjection methods, and embryonic stem cell methods. The procedures for manipulation of the rodent embryo and for microinjection of DNA are described in detail in Hogan et al., Manipulating the Mouse Embryo (Cold Spring Harbor Laboratory, Cold Spring Harbor, N.Y., 1986), and U.S. Pat. No. 6,201,165, the teachings of which are generally known and are incorporated herein.

Treatment of animals with test compounds (e.g., crystallin agonists) involve the administration of the compound, in an appropriate form, to the animal. Administration is by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated are ophthalmic administration, for example, it is contemplated that all local routes to the eye may be used, including topical, subconjunctival, periocular, retrobulbar, subtenon, intracameral, intravitreal, intraocular, subretinal, posterior or anterior juxtascleral, and suprachoroidal administration.

E. Production of Agonist

In an extension of any of the previously described screening assays, the present invention also provides for methods of producing or manufacturing crystallin agonists. The methods comprising any of the preceding screening steps followed by an additional step of “producing or manufacturing the candidate substance identified as an agonist of crystallin” the screened activity. Manufacturing can entail any well known and standard technique used by those of skill in the art, such as synthesizing the compound and/or deriving the compound from a natural source.

### IV. Treatment/Prevention

In certain aspects of the present invention, compounds are used to treat and/or prevent optic nerve damage. More particularly, the compounds are used to increase or stimulate the expression and/or activity of a crystallin gene or protein in ocular tissue for example, retinal tissue and the optic nerve. Stimulation of up-regulation of crystallin expression will delay the progression of disease, decrease optic nerve damage, decrease retinal damage, etc.

Types of optic nerve damage that may be treated and/or prevented using the compounds of the present invention can include, for example, glaucoma and other optic neuropathies. Glaucoma more specifically includes primary open angle glaucoma, acute angle closure glaucoma, normal tension glaucoma, low tension glaucoma, ocular hypertension. Optic neuropathies that may be treated and/or prevented by the present invention may include for example, ischemic optic neuropathies, such as anterior ischemic optic neuropathy and optic neuropathies associated with vascular disease such as diabetes.

Treatment and/or prevention methods will involve treating an individual with an effective amount of a composition containing a crystallin agonist or related-compound thereof. An effective amount is described, generally, as that amount sufficient to detectably and repeatedly to ameliorate, reduce, minimize or limit the extent of a disease or its symptoms. More specifically, it is envisioned that the treatment with crystallin agonist or related-compounds thereof will stabilize or improve visual function (as measured by visual acuity, visual field, or other method known to those of ordinary skill in the art), decrease retina deterioration, decrease the severity of glaucoma, and/or delay or prevent the onset of optic nerve damage resulting from glaucoma.

An effective amount of a crystallin agonist that may be administered to a cell includes a dose of about 0.1 μM to about 100 μM. More specifically, doses of a crystallin agonist to be administered are from about 0.1 μM to about 10 μM; about 1 μM to about 5 μM; about 5 μM to about 10 μM; about 10 μM to about 15 μM; about 15 μM to about 20 μM; about 20 μM to about 30 μM; about 30 μM to about 40 μM; about 40 μM to about 50 μM; about 50 μM to about 60 μM; about 60 μM to about 70 μM; about 70 μM to about 80 μM; about 80 μM to about 90 μM; and about 90 μM to about 100 μM. Of course, all of these amounts are exemplary, and any amount in-between these points is also expected to be of use in the invention.

In further embodiments, an effective amount of a crystallin agonist that may be administered to a subject includes a dose from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

Furthermore, the compounds can be used to prevent the onset or delay the onset or reduce the severity of glaucoma. For example, a subject may not exhibit any clinical symptoms, but may have a family history or several risk factors for the development of glaucoma. Thus, the crystallin agonists of the present invention can prevent the onset, delay the onset or reduce the severity of glaucoma in the subject.

Thus, a subject can be a subject who is known or suspected of being free of a particular disease or health-related condition at the time the relevant agent is administered. The subject, for example, can be a subject with no known disease or health-related condition (i.e., a healthy subject). In some embodiments, the subject is a subject at risk of developing a particular disease or health-related condition. Thus, in certain embodiments of the invention, methods include identifying a patient in need of treatment. A patient may be identified, for example, based on taking a patient history, or based on findings on clinical examination.

### V. Combination Treatments

In order to increase the effectiveness of the methods of the present invention, it may be desirable to combine the crystallin agonists with standard glaucoma treatments known and used by those of skill in the art.

In certain embodiments, one would generally administer to the subject a crystallin agonist in combination with an additional therapeutic agent. These compositions would be provided in a combined amount effective to reduce intraocular pressure, increase visual function, decrease deterioration of the retina, etc. This process may involve administering the crystallin agonist in combination with an additional therapeutic agent or factor(s) at the same time. This may be achieved by administering with a single composition or pharmacological formulation that includes both agents, or by administering two distinct compositions or formulations, at the same time, wherein one composition includes the crystallin agonists and the other includes the additional agent.

Alternatively, treatment with crystallin agonists may precede or follow the additional agent treatment by intervals ranging from seconds, to minutes, to weeks to months, to years. In embodiments where the additional agent is applied separately, one would generally ensure that a significant period of time did not expire between the time of each delivery, such that the agent would still be able to exert an advantageously combined effect. In such instances, it is contemplated that administer with both modalities within minutes to hours. In some situations, it may be desirable to extend the time period for treatment significantly, however, where several days (2, 3, 4, 5, 6 or 7) to several weeks (1, 2, 3, 4, 5, 6, 7 or 8) to several months (1, 2, 3, 4, 5, 6) lapse between the respective administrations.

A. Pharmaceutical Treatments

Examples of pharmacological agents to treat glaucoma that can be used in combination with the crystallin agonists of the present invention include beta-blockers, such as timolol and betaxolol, and carbonic anhydrase inhibitors, such as dorzolamide and brinzolamide. Other agents may also include, prostaglandin analogs, which are believed to reduce intraocular pressure by increasing uveoscleral outflow. Three marketed prostaglandin analogs are latanoprost, bimatoprost and travoprost. Additional pharmacological agents that can be used in combination with the crystalline agonists of the present invention include alpha-I antagonists (e.g., nipradolol), alpha-2 agonists (e.g., iopidine and brimonidine), miotics (e.g., pilocarpine and epinephrine), hypotensive lipids (e.g., bimatoprost and compounds set forth in U.S. Pat. No. 5,352,708), neuroprotectants (e.g., memantine), serotonergics [e.g., 5-HT₂ agonists, such as S-(+)-1-(2-aminopropyl)-indazole-6-ol)], anti-angiogenesis agents (e.g., anecortave acetate), and ethacrynic acid

These pharmaceutical agents are typically administered topically, and work to either reduce aqueous production or they act to increase outflow.

B. Surgical Treatments

In addition to pharmacological agents, surgical procedures can be performed in combination with the administration of the crystallin agonists. One such surgical procedure can include, laser trabeculoplasty. In laser trabeculoplasty, energy from a laser is applied to a number of noncontiguous spots in the trabecular meshwork. It is believed that the laser energy stimulates the metabolism of the trabecular cells, and changes the extracellular material in the trabecular meshwork.

Another surgical procedure may include filtering surgery. With filtering surgery, a hole is made in the sclera near the angle. This hole allows the aqueous fluid to leave the eye through an alternate route. The most commonly performed filtering procedure is a trabeculectomy. In a trabeculectomy, a conjunctiva incision is made, the conjunctiva being the transparent tissue that covers the sclera. The conjunctiva is moved aside, exposing the sclera at the limbus. A partial thickness scleral flap is made and dissected half-thickness into the cornea. The anterior chamber is entered beneath the scleral flap and a section of deep sclera and/or trabecular meshwork is excised. The scleral flap is loosely sewn back into place. The conjunctival incision is tightly closed. Post-operatively, the aqueous fluid passes through the hole, beneath the scleral flap which offers some resistance and collects in an elevated space beneath the conjunctiva called a bleb. The fluid then is either absorbed through blood vessels in the conjunctiva or traverses across the conjunctiva into the tear film.

### VI. Pharmaceutics and Formulations

A. Dosage

The phrase “pharmaceutically effective amount” is an art-recognized term, and refers to an amount of an agent that, when incorporated into a pharmaceutical composition of the present invention, produces some desired effect at a reasonable benefit/risk ratio applicable to any medical treatment. In certain embodiments, the term refers to that amount necessary or sufficient to increase the expression or activity of crystallin. The effective amount may vary depending on such factors as the disease or condition being treated, the particular composition being administered, or the severity of the disease or condition. One of skill in the art would be familiar with determining an effective amount of a particular agent without necessitating undue experimentation.

The phrase “pharmaceutically acceptable” is art-recognized and refers to compositions, polymers and other materials and/or dosage forms which are suitable for use in contact with the tissues of human beings and animals without excessive toxicity, irritation, allergic response, or other problem or complication, commensurate with a reasonable benefit/risk ratio as determined by one of ordinary skill in the art.

The amount of agent or compound to be included in the compositions or applied in the methods set forth herein will be whatever amount is pharmaceutically effective and will depend upon a number of factors, including the identity and potency of the chosen agent or compound. One of ordinary skill in the art would be familiar with factors that are involved in determining a pharmaceutically effective dose of an agent or compound.

In particular embodiments, the composition is administered once a day. However, the compositions of the present invention may also be formulated for administration at any frequency of administration, including once a week, once every 5 day, once every 3 days, once every 2 days, twice a day, three times a day, four times a day, five times a day, six times a day, eight times a day, every hour, or any greater frequency. One of ordinary skill in the art would be familiar with establishing a therapeutic regimen. Factors involved in this determination include the disease to be treated, particular characteristics of the subject, and the delivery method used.

B. Formulations

Regarding the methods set forth herein, compositions containing crystallin agonists can be formulated in any manner known to those of ordinary skill in the art. In the compositions set forth herein, the concentration of the crystallin agonists can be any concentration known or suspected by those of ordinary skill in the art to be of benefit in the treatment of optic nerve damage.

The actual dosage amount of a composition of the present invention administered to a subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain non-limiting embodiments, the pharmaceutical compositions may comprise, for example, at least about 0.1%, by weight or volume, of an active ingredient. In other embodiments, the active ingredient may comprise between about 2% to about 75% of the weight or volume of the unit, or between about 25% to about 60%, and any range derivable therein.

In certain embodiments of the present invention, the compositions set forth herein include more than one crystallin agonist. One of ordinary skill in the art would be familiar with preparing and administering pharmaceutical compositions that include more than one therapeutic agent. In some embodiments, the composition includes one or more additional therapeutic agents that are not crystallin agonists.

In addition to the crystallin agonists, the compositions of the present invention optionally comprise one or more excipients. Excipients commonly used in pharmaceutical compositions include, but are not limited to, carriers, tonicity agents, preservatives, chelating agents, buffering agents, surfactants and antioxidants.

A person of ordinary skill will recognize that the compositions of the present invention can include any number of combinations of ingredients (e.g., active agent, polymers, excipients, etc.). It is also contemplated that that the concentrations of these ingredients can vary. For example, in one-non-limiting aspect, a composition of the present invention can include at least about 0.0001% to about 0.001%, 0.001% to about 0.01%, 0.01% to about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the ingredients mentioned throughout the specification and claims. In non-limiting aspects, the percentage can be calculated by weight or volume of the total composition. A person of ordinary skill in the art would understand that the concentrations can vary depending on the addition, substitution, and/or subtraction of ingredients in a given composition.

The phrase “pharmaceutically acceptable carrier” is art-recognized, and refers to, for example, pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body. Each carrier must be “acceptable” in the sense of being compatible with the other ingredients of the supplement and not injurious to the patient.

Any of a variety of carriers may be used in the formulations of the present invention including water, mixtures of water and water-miscible solvents, such as C1-C7-alkanols, vegetable oils or mineral oils comprising from 0.5 to 5% non-toxic water-soluble polymers, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, mixtures of those polymers. The concentration of the carrier is, typically, from 1 to 100000 times the concentration of the active ingredient.

Suitable tonicity-adjusting agents include mannitol, sodium chloride, glycerin, sorbitol and the like. Suitable preservatives include p-hydroxybenzoic acid ester, benzalkonium chloride, benzododecinium bromide, polyquaternium-1 and the like. Suitable chelating agents include sodium edetate and the like. Suitable buffering agents include phosphates, borates, citrates, acetates and the like. Suitable surfactants include ionic and nonionic surfactants, though nonionic surfactants are preferred, such as polysorbates, polyethoxylated castor oil derivatives and oxyethylated tertiary octylphenol formaldehyde polymer (tyloxapol). Suitable antioxidants include sulfites, ascorbates, BHA and BHT. The compositions of the present invention optionally comprise an additional active agent.

In particular embodiments, the compositions are suitable for application to mammalian eyes. For example, for ophthalmic administration, the formulation may be a solution, a suspension, a gel, or an ointment.

In preferred aspects, the compositions that include crystallin agonists will be formulated for topical application to the eye in aqueous solution in the form of drops. The term “aqueous” typically denotes an aqueous composition wherein the carrier is to an extent of >50%, more preferably >75% and in particular >90% by weight water. These drops may be delivered from a single dose ampoule which may preferably be sterile and thus rendering bacteriostatic components of the formulation unnecessary. Alternatively, the drops may be delivered from a multi-dose bottle which may preferably comprise a device which extracts preservative from the formulation as it is delivered, such devices being known in the art.

In other aspects, components of the invention may be delivered to the eye as a concentrated gel or similar vehicle which forms dissolvable inserts that are placed beneath the eyelids.

The compositions of the present invention are preferably not formulated as solutions that undergo a phase transition to a gel upon administration to the eye.

In addition to the one or more crystallin agonists, the compositions of the present invention may contain other ingredients as excipients. For example, the compositions may include one or more pharmaceutically acceptable buffering agents, preservatives (including preservative adjuncts), non-ionic tonicity-adjusting agents, surfactants, solubilizing agents, stabilizing agents, comfort-enhancing agents, polymers, emollients, pH-adjusting agents and/or lubricants.

For topical formulations to the eye, the formulation are preferably isotonic, or slightly hypotonic in order to combat any hypertonicity of tears caused by evaporation and/or disease. The compositions of the present invention generally have an osmolality in the range of 220-320 mOsm/kg, and preferably have an osmolality in the range of 235-260 mOsm/kg. The compositions of the invention have a pH in the range of 5-9, preferably 6.5-7.5, and most preferably 6.9-7.4.

The formulations set forth herein may comprise one or more preservatives. Examples of preservatives include quaternary ammonium compounds, such as benzalkonium chloride or benzoxonium chloride. Other examples of preservatives include alkyl-mercury salts of thiosalicylic acid, such as, for example, thiomersal, phenylmercuric nitrate, phenylmercuric acetate or phenylmercuric borate, sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, or sorbic acid.

In certain embodiments, the crystallin agonists are formulated in a composition that comprises one or more tear substitutes. A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxy methylcellulose sodium and hydroxy propylcellulose; dextrans such as dextran 70; water soluble proteins such as gelatin; vinyl polymers, such as polyvinyl alcohol, polyvinylpyrrolidone, and povidone; and carbomers, such as carbomer 934P, carbomer 941, carbomer 940 and carbomer 974P. The formulation of the present invention may be used with contact lenses or other ophthalmic products.

In some embodiments, the compositions set forth herein have a viscosity of 0.5-10 cps, preferably 0.5-5 cps, and most preferably 1-2 cps. This relatively low viscosity insures that the product is comfortable, does not cause blurring, and is easily processed during manufacturing, transfer and filling operations.

C. Route of Administration

In the methods set forth herein, administration to a subject of a pharmaceutically effective amount of a composition that includes one or more crystallin agonists by any method known to those of ordinary skill in the art.

For example, the composition may be administered locally, topically, intravenously, intradermally, intraarterially, intraperitoneally, intrapleurally, intratracheally, intranasally, intravitreally, intramuscularly, subcutaneously, subconjunctival, intravesicularlly, mucosally, intraumbilically, intraocularly, orally, by inhalation, by injection, by infusion, by continuous infusion, by localized perfusion.

In particular embodiments, the composition is administered topically to the ocular surface. Regarding ophthalmic administration, it is contemplated that all local routes to the eye may be used, including topical, subconjunctival, periocular, retrobulbar, subtenon, intracameral, intravitreal, intraocular, subretinal, posterior or anterior juxtascleral, and suprachoroidal administration.

### VII. Therapeutic Kits

Any of the compositions described herein may be comprised in a kit. Therapeutic kits of the present invention are kits comprising a crystallin agonist or a related-compound thereof. Such kits will generally contain, in suitable container means, a pharmaceutically acceptable formulation of a crystallin agonist or related-compound thereof. The kit may have a single container means, and/or it may have distinct container means for each compound.

When the components of the kit are provided in one and/or more liquid solutions, the liquid solution is an aqueous solution, with a sterile aqueous solution being particularly preferred. The crystallin agonist compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the crystallin agonist is suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Irrespective of the number and/or type of containers, the kits of the invention may also comprise, and/or be packaged with, an instrument for assisting with the injection/administration and/or placement of the crystallin agonist composition within the body of an animal. Such an instrument may be a syringe, pipette, forceps, and/or any such medically approved delivery vehicle.

### VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Changes in Crystallin Gene Expression

Standard methods were used to determine whether crystallin is involved in glaucomatous optic neuropathy (Jia et al., 2000).

Briefly, chronic elevation of rat intraocular pressure (IOP) leading to optic nerve damage is induced by episcleral injection of hypertonic saline, which causes sclerosis and blocks aqueous humor outflow pathways. Expression of crystallin in the retina and optic nerve head (ONH) was evaluated using Affymetrix gene chips. Table 1 shows qualitative analysis of gene expression (log2 change) correlated with the degree of glaucomatous optic nerve damage. Quantitative PCR (Q-PCR) and immunohistochemistry are performed to confirm the gene array data.

TABLE 1

| | | | Mild | Severe |
|---|---|---|---|---|
| Gene | Accession # | No Damage | Damage | Damage |
| CRYAA | U47921 | | −1.6 | −2.1 |
| CRYAB | X60351 | −1.2 | −1.2 | −1.1 |
| CRYBA1 | rc_AI072996 | | −1.5 | |
| CRYBA2 | rc_AI07317 | | −1.5 | −1.1 |
| CRYBA3 | AF013248 | −1.1 | | |
| CRYBA1/3 | X15143 | −1.2 | −2 | −1 |
| CRYBA4 | AF013247 | | −1.7 | |
| CRYBB2 | X16072 | −1.1 | −1.7 | −1.4 |
| CRYGS | rc_AI112249 | | −1.7 | |

### Example 2

### Efficacy Evaluation in Rodent Model of Glaucoma

A rat model of glaucoma is induced by injection of hypertonic saline into an episcleral vein generating elevated IOP, as described in Example 1. Specific crystallin antagonists are administered to the eye of these ocular hypertensive rats. Efficacy is determined by quantifying the number of retinal ganglion cells in retinal whole mounts and by examination of cross-sectioned optic nerves for axonal damage.

### Example 3

### Ocular Safety Evaluation in New Zealand Albino Rabbits

For example, both eyes of New Zealand albino rabbits are dosed with one 30 μL aliquot of a test crystallin agonist in a vehicle. Animals are monitored continuously from 0.5 hr post-dose out to days (depending on the agent and route of administration) or until effects are no longer evident.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
Alge, Invest Opthalmol V is Sci. 2002 November; 43(11):3575-82
Atschul, S. F. et al., J. Molec. Biol., 215, 403 (1990).
Biocomputing: Informatics and Genome Projects, Smith, D. W., ed., Academic Press, New York, 1993.
Bohmann et al., (1989) Cell. November 17; 59(4):709-17.
Carillo, H., and Lipman, D., SIAM J Applied Math., 48:1073 (1988).
Chatterjee, et al., (1995) Ann. N.Y. Acad. Sci., 770:79-90.
Kaplitt et al., (1994) Nat'l Genet., 8:148-153.
Computational Molecular Biology, Lesk, A. M., ed., Oxford University Press, New York, 1988.
Computer Analysis of Sequence Data, Part I, Griffin, A. M., and Griffin, H. G., eds., Humana Press, New Jersey, 1994.
Courey et al., (1988) Cell. 55(5):887-98.
D. Bennett et al., Journal of Molecular Recognition, 8: 52-58 (1995).
Devereux, J., et al., Nucleic Acids Research, 12(1), 387 (1984).
Friedman et al., (1990) Genes Dev. August; 4(8):1416-26.
Gerster et al., (1990) EMBO J. (5):1635-43.
Hope et al., (1987) EMBO J. September; 6(9):2781-4.
Jia et al., Investigative Opthalmology and Visual Science. 2000; 41:1380-1385.
Jones et al., Neuroreport. 1998 Dec. 21; 9(18):4161-5.
K. Johanson et al., The Journal of Biological Chemistry, 270, (16): 9459-9471 (1995).
Kantorow, Int J Biol Macromol. 1998 May-June; 22(3-4):307-14.
Li et al., Exp Cell Res. 2001 Jun. 10; 266(2):279-91.
Ma et al., (1987) 50:137-42.
Magabo et al., Invest Opthalmol V is Sci. 2000 September; 41(10):3056-60
Martin et al., (1990) Genes Dev. (11): 1886-98.
Samulski et al., (1987) J. Virol., 61(10):3096-3101.
Mermod et al., (1989) Cell. 58(4):741-53.
Muller-Immergluck et al., (1990) EMBO J. 9(5):1625-34.
Sadowski et al., (1988) Nature. 335(6190):563-4.
Sequence Analysis in Molecular Biology, von Heinje, G., Academic Press, 1987.
Ridgeway, (1988) In: Vectors: A survey of molecular cloning vectors and their uses, pp. 467-492.
Sequence Analysis Primer, Gribskov, M. and Devereux, J., eds., M Stockton Press, New York, 1991.

What is claimed is:

1. A method of manufacturing a crystallin agonist comprising:
(a) providing a candidate substance suspected of increasing crystallin expression or activity in ocular tissue;
(b) selecting the crystallin agonist by assessing the ability of the candidate substance to increase crystallin expression or activity in ocular tissue; and
(c) manufacturing the selected crystallin agonist.

2. The method of claim 1, wherein the candidate substance is a small molecule, a protein, or a nucleic acid molecule.

3. The method of claim 1, wherein the providing step is further defined as providing in a cell or a cell-free system a crystallin polypeptide and the crystallin polypeptide is contacted with the candidate substance.

4. The method of claim 3, wherein the crystallin polypeptide is selected from the group consisting of SEQ. ID. NO. 1, SEQ. ID. NO. 2, SEQ. ID. NO. 3, SEQ. ID. NO. 4, SEQ. ID. NO. 5, SEQ. ID. NO. 6, SEQ. ID. NO. 7, SEQ. ID. NO. 8, and SEQ. ID. NO. 9.

5. The method of claim 1, wherein the providing step is further defined as providing a nucleic acid molecule that encodes the crystallin polypeptide.

6. The method of claim 5, wherein the nucleic acid molecule is selected from the group consisting of CRYAA, CRYAB, CRYBB, CRYBA, CRYGS, and CRYM.

7. A pharmaceutical composition comprising an agonist made according to any one of claims 1-6 admixed with a pharmaceutical carrier.

8. A method of treating and/or preventing optic nerve damage comprising administering to a subject an effective amount of a crystallin agonist admixed with a pharmaceutical carrier, wherein said amount increases expression and/or activity of the crystallin in ocular tissue thereby treating and/or preventing optic nerve damage.

9. The method of claim 8, wherein said optic nerve damage is glaucoma.

10. The method of claim 9, wherein said glaucoma is primary open angle glaucoma.

11. The method of claim 8, wherein said optic nerve damage is an optic neuropathy.

12. The method of claim 8, wherein administering is topical.

13. The method of claim 8, wherein the subject has increased intraocular pressure in at least one eye.

14. The method of claim 8, wherein ocular tissue is retinal tissue or optic nerve tissue.

15. An expression vector comprising:
a) a nucleic acid sequence selected from the group consisting of SEQ. ID. NO. 10, SEQ. ID. NO. 11, SEQ. ID. NO. 12, SEQ. ID. NO. 13, SEQ. ID. NO. 14, SEQ. ID. NO. 15, SEQ. ID. NO. 16, SEQ. ID. NO. 17 and SEQ. ID. NO. 18; or
b) an isolated polynucleotide sequence encoding a protein, wherein said protein is selected from the group consisting of:
(1) a polynucleotide sequence encoding a sequence selected from the group consisting of SEQ. ID. NO. 1, SEQ. ID. NO. 2, SEQ. ID. NO. 3, SEQ. ID. NO. 4, SEQ. ID. NO. 5, SEQ. ID. NO. 6, SEQ. ID. NO. 7, SEQ. ID. NO. 8, and SEQ. ID. NO. 9;
(2) a polynucleotide sequence encoding an amino acid sequence having at least 80% identity with a sequence selected from the group consisting of SEQ. ID. NO. 1, SEQ. ID. NO. 2, SEQ. ID. NO. 3, SEQ. ID. NO. 4, SEQ. ID. NO. 5, SEQ. ID. NO. 6, SEQ. ID. NO. 7, SEQ. ID. NO. 8, and SEQ. ID. NO. 9;
(3) an isolated nucleic acid molecule that hybridizes with the polynucleotide sequence of (1) under hybridization conditions of 0.02 M to about 0.15 M NaCl at temperatures of about 50° C. to about 70° C.; and
(4) an isolated polynucleotide sequence that is complementary to (1), (2) or (3).

16. The expression vector of claim 15, wherein the expression vector is further defined as a viral or plasmid vector.

17. The expression vector of claim 16, wherein the viral vector is an adenoviral vector, an adeno-associated viral vector, a retroviral vector, a lentiviral vector, a herpes viral vector, polyoma viral vector or hepatitis B viral vector.

18. The expression vector of claim 15, wherein the expression vector is comprised in a non-viral delivery system.

19. The expression vector of claim 18, wherein the non-viral delivery system comprises one or more lipids.
